(19) United States
(12) Patent Application Publication
(10) Pub. No.: US 2024/0024695 A1
(43) Pub. Date: Jan. 25, 2024

(54) Methods of Treating Tinnitus Using Noninvasive Light Therapy

(71) Applicant: Erchonia Corporation, LLC, Melbourne, FL (US)

(72) Inventors: Steven C SHANKS, Melbourne, FL (US); Travis SAMMONS, Melbourne, AZ (US)

(73) Assignee: Erchonia Corporation, LLC, Melbourne, AZ (US)

(21) Appl. No.: 17/870,587

(22) Filed: Jul. 21, 2022

(51) Int. Cl. A61N 5/06; A61N 5/067

(52) U.S. Cl. CPC A61N 5/0613; A61N 5/067; A61N 2005/0663

(57) ABSTRACT

Light energy is applied externally to the ear of a person suffering tinnitus and down the side of the person's neck below that ear. If both ears suffer tinnitus, then the other ear is treated in the same manner. The wavelength of the applied light energy is in the visible violet-blue range of about 400-500 nm. Preferably applied light energy is 405 nm±10 nm. The light energy is preferably laser energy and is supplied by a laser device that emits a line of light energy. The line of light energy is waved manually across a treatment area in a continuous, sweeping manner from about one inch above the person's first ear down the side of the person's neck below the first ear. Preferably the light energy is applied using a hand-held device so that a person can treat him or herself at home.

### FIELD OF INVENTION

This invention relates generally to methods for treating tinnitus. This invention relates more particularly to non-invasive tinnitus treatments using light therapy.

### BACKGROUND

Tinnitus is a phantom ringing sound a person hears in his or her ears. Tinnitus manifests as other sounds, too, variously described as buzzing, roaring, water rushing, whooshing, clicking, hissing, humming or a squeal, in one or both ears. Tinnitus is a common problem: it affects about 15% to 20% of people and is especially common in older adults. Treatments include sound therapy, hearing aids, and wearable sound masking devices. Despite being so widespread, there is no cure for tinnitus.

The noise heard by those with tinnitus isn't caused by an external sound, but the causes of tinnitus are not well understood and in many cases an exact cause is never found. A number of health conditions can cause or worsen tinnitus. For example, hearing loss can cause tinnitus, as can head or neck injuries. Some medications are known to cause tinnitus due to ototoxicity, including nonsteroidal anti-inflammatory drugs such as aspirin, and certain antibiotics, cancer drugs, diuretics, antimalarial drugs and antidepressants.

Low-level laser therapy (“LLLT”) has been shown through numerous clinical studies and regulatory clearances to be a safe, effective, simple, non-invasive and side-effect free alternative to medication and surgical procedures for the reduction of symptoms in a variety of conditions. Because of the low power level, typically below 500 mW depending on the target tissue, LLLT treatment causes no evident temperature rise in the treated tissue and therefore no significant change in the gross tissue structure. LLLT differs from other light-based treatments because it does not ablate and is not based on heating. LLLT is not thermal radiation and instead works through photobiomodulation, which is light-induced photochemical reactions in biological systems. For example, Vitamin D synthesis in human skin is an example of a photochemical reaction. When the ultraviolet B wavelength in sunlight strikes human skin, it converts a universally present form of cholesterol, 7-dehydrocholesterol, to vitamin D₃. LLLT is therefore is also referred to as photobiomodulation therapy (“PBMT”).

It is an object of this invention to apply light energy to a person to treat tinnitus. It is another object of this invention to provide a non-invasive, non-contact non-thermal method of treating tinnitus that can be self-administered by a person with tinnitus.

### SUMMARY OF THE INVENTION

Noninvasive, non-contact methods are disclosed for treating a person who suffers tinnitus in one or both ears. Light energy is applied externally to the ear suffering tinnitus and down the side of the person's neck below that ear. If both ears suffer tinnitus, then the other ear is treated in the same manner. The wavelength of the applied light energy is in the visible violet-blue range of about 400-500 nm. Preferably applied light energy is 405 nm±10 nm. The light energy is preferably laser energy and is supplied by a laser device that emits a line of light energy. The line of light energy is waved manually across a treatment area in a continuous, sweeping manner from about one inch above the person's first ear down the side of the person's neck below the first ear. Preferably the light energy is applied using a hand-held, battery powered device so that a person can treat him or herself at home.

### BRIEF DESCRIPTION OF THE DRAWING

FIG. 1 is a side view illustrating light energy being applied to a person's head using a hand-held device and the approximate treatment area for treating the person's left ear which suffers tinnitus.

### DETAILED DESCRIPTION OF THE INVENTION

Noninvasive, non-contact methods are disclosed for treating a person who suffers tinnitus in one or both ears. The person referred to herein may be any gender, although for consistency is referred to herein using male pronouns.

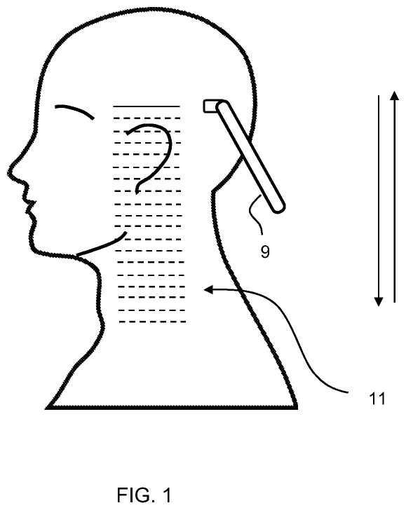

Light energy 11 is applied externally to the person at least at the side of the person's neck below the ear suffering tinnitus some distance down to about the collar bone. In addition to applying light energy to the neck, light energy may also be applied to the ear suffering tinnitus. In addition to applying light energy to the neck and ear suffering tinnitus, light energy can also be applied to the area about 1″ above the ear. When used to describe the treatment area, “about” is known to a person of skill in the art of light therapy. In a preferred method, light energy is applied to the person starting about 1″ above the ear suffering tinnitus, to the ear, and down the side of the person's neck below that ear all the way to the person's collar bone. See FIG. 1 which illustrates a line of laser light at different locations as the light is swept across the person's head and neck.

The light energy is applied in a continuous sweeping motion and the light-emitting device 9 does not contact the person. While applying treatment some of the light may enter the ear canal, however the method does not intentionally deliver light energy to the ear canal. Due to the shape or the ear canal, it is unlikely the photons would directly stimulate the ear drum, although the photochemical effects created as the light triggers photobiomodulation may reach the ear drum and deeper into the inner ear as well as affect the automatic nervous system. It is interesting to note that tinnitus and hearing loss are separate, uncorrelated issues, even though they both involve a person's ear. Most of the people that have tinnitus do not have hearing issues, and persons with hearing issues do not always have tinnitus. As disclosed in the prior art, light therapy for treating hearing loss does not apply light energy to the ear and side of neck

There are a number of variables in light therapy, including the wavelength of the light, the power of the light source, the area impinged by the light, the shape of the beam spot when the light impinges the treated area, the pulse frequency, the intensity or fluence of the light energy, and the treatment duration and frequency. The success of each therapy depends on the relationship and combination of these variables with the tissue characteristics of the specific person and desired outcome.

It is a long-held belief by experts in the field that only long, near infrared wavelengths can penetrate deep enough into a person's tissue or bone to affect cellular behavior, and that shorter wavelengths cannot do so. However shorter wavelengths do have therapeutic effects because, at least in part, they trigger photobiomodulation. Violet light has more energy per photon than red and infrared, and allows for greater electron transfer and consequent photochemical effects at greater depth. Advantageously, cool colors including the violet-blue wavelengths excite the parasympathetic system, which plays a role in tinnitus.

The wavelength of the applied light energy used to treat tinnitus is in the visible violet-blue range of about 390-500 nm. Commercial semiconductor laser diodes and other light energy emitters have a spread of ±10 nm from nominal. A person skilled in the art would recognize that “about” means±10 nm from the stated wavelength. For example, if a 405 nm wavelength is specified for treatment, a laser denoted as a 400 nm laser would suffice, because 400 nm is within the spread from nominal. Preferably the applied light energy 405 nm±10 nm. In some embodiments multiple wavelengths are used, either in series, alternately, or simultaneously.

The light can be from any source including light-emitting diodes, hard-wired lasers, or laser diodes, but preferably is from one or more semiconductor laser diodes. The light can be emitted from the same light emitter or from multiple emitters. The light energy can be applied to the person using a variety of devices, such as a hand-held light-emitting device, a full-body scanner, a wall-mounted light-emitting device, or a stand-alone light-emitting device. The light can be applied by a person other than the person with tinnitus, such as an audiologist, physical therapist, physician, nurse, spouse or other care-giver. In some embodiments, the therapist may prescribe a protocol for the person and the person can self-administer the protocol to treat his tinnitus. Preferably the light energy is applied using a hand-held device so that a person can treat himself. Preferably the light is emitted as a line from a hand-held laser device, as shown in U.S. Pat. Nos. 6,746,473, 7,118,588 or 7,947,067 which are incorporated herein by reference. The line is waved manually across a person's tissue in the targeted area in a continuous, sweeping manner.

In other embodiments the shape of the beam spot on the treated area is an apparent circle, which is actually a rotating diameter by a line of light. U.S. Pat. No. 7,922,751, incorporated herein by reference, discloses a device to sweep such a circular beam spot. The device disclosed in that patent can be programmed to move the scanning head in a manner to achieve any desired shape of a treatment zone on the head of a person. A sample selection of available scan patterns is shown in that patent at FIGS. 8a-h.

The applied light is low power, often less than 7.5 mW and typically from emitters of less than 1 W. For example, 1 mW, 3 mW, 5 mW, and 7.5 mW of power may be used. In some embodiments, two emitters at 7.5 mW each are used, for a total of 15 mW. This low-level light therapy has an energy dose rate that causes no immediate or long term detectable temperature rise of the treated tissue and no macroscopically visible changes in tissue structure. Consequently, the tissue impinged by the light is not heated and is not damaged.

The applied light energy is applied with a pulse frequency or frequencies from 0 to 100,000 Hz. Preferably the pulse frequency is at 8, 9, 10, or 11 Hz.

Light energy is applied to the person's head 11. See FIG. 1. The light energy is preferably applied directly to the person's skin, but may be applied through the person's hair. Because the light energy is low level, there is no need for an intervening device or structure used between where the laser light exits the laser device and the person to cool, alter, dissipate, or otherwise affect the transmission of the light energy to the person or affect the temperature of the treated surface, the treated tissue, tissue within the treatment area, around the treatment area, or any intervening or targeted tissue within the person's body.

Typically the person is treated while the person is vertical or nearly vertical, as opposed to prone or supine. Usually the person is seated in a chair or standing, often in front of a mirror.

The frequency and duration of each treatment session will vary depending on a number of factors including the severity of the tinnitus. Typically a treatment is 1-5 minutes long and a treatment is applied daily to the affected ear or ears until the tinnitus goes away or is relieved to a degree that makes the person not notice it. In a preferred embodiment the light energy is applied to the affected side of the head and neck for 5 minutes each day for four weeks.

Advantageously, these methods use low-level light therapy which has an energy dose rate that causes no detectable temperature rise of the treated tissue immediately upon treatment or over time, and no macroscopically visible changes. This holds true at the treated surface, the treated tissue, tissue within the treatment area, around the treatment area, or any intervening or targeted tissue of the person's body. No cooling or blanching of tissue is needed to maintain the tissue temperature, nor is any diffusion of the treatment energy needed to maintain tissue temperature. Consequently, the light energy is applied directly to the person, with no intervening device or structure between where the laser light exits the laser device and the person, making for a relatively simple and efficient treatment process. In other words, no element exists between the light once it is emitted from the laser device until it impinges on the person, so there is nothing in the line of emission that would change the direction of the emission, stop it, or modulate it.

### Example 1—Self-Administered Tinnitus Treatment

A person suffers tinnitus in his right ear. An audiologist prescribes the application of low-level laser energy to the person's right ear and neck below the right ear using a hand-held laser having a wavelength of 405 nm±10 nm, for 5 minutes each day for four weeks. The person obtains a battery-powered, hand-held laser of the appropriate parameters and treats himself as prescribed.

### Example 2—Second-Party Administered Tinnitus Treatment

A person suffers tinnitus in both ears. An audiologist prescribes the application of low-level laser energy to the person's ears and neck below the each ear using a hand-held laser having a wavelength of 405 nm±10 nm, for 5 minutes each day for four weeks. The person's spouse uses a battery-powered, hand-held laser of the appropriate parameters and treats the person as prescribed.

While there has been illustrated and described what is at present considered to be the preferred embodiments of the present invention, it will be understood by those skilled in the art that various changes and modifications may be made and equivalents may be substituted for elements thereof without departing from the true scope of the invention. Therefore, it is intended that this invention not be limited to the particular embodiments disclosed, but that the invention will include all embodiments falling within the scope of the appended claims.

1. A method of treating a person who suffers tinnitus in one or both ears, the method comprising:
a. applying visible light energy between 395-500 nm externally to the side of the person's neck below the person's first ear that suffers tinnitus.

2. The method of claim 1 wherein the light energy comprises light energy at 405 nm.

3. The method of claim 1 wherein the light energy consists of light at 405 nm±10 nm.

4. The method of claim 1 further comprising applying the visible light energy externally to the first ear.

5. The method of claim 1 wherein the light energy is laser energy.

6. The method of claim 5 wherein the direct application of the laser energy to the person causes no detectable temperature rise of the treated area.

7. The method of claim 5 wherein the laser energy is applied to the person using a laser device that emits a line of laser energy and the line is swept across the treatment area in a continuous sweeping motion.

8. The method of claim 7 wherein the laser device is hand-held.

9. A method of treating a person who suffers tinnitus in one or both ears, the method comprising:
a. applying visible light energy between 395-500 nm externally to the person using a device that emits a line of light energy, wherein the light energy is applied in a continuous sweeping motion from about one inch above the person's first ear down the side of the person's neck below the first ear.

10. The method of claim 9 wherein the light energy comprises light energy at 405 nm.

11. The method of claim 9 wherein the light energy consists of light at 405 nm±10 nm.

12. The method of claim 9 wherein the light energy is laser energy.

13. The method of claim 12 wherein the direct application of the laser energy to the person causes no detectable temperature rise of the treated area.

14. The method of claim 9 wherein the light device is hand-held.

15. A method for a person who suffers tinnitus in one or both ears to treat himself, the self-treatment method comprising:
a. applying visible light energy between 400-500 nm externally to himself from about one inch above his first ear down the side of his neck below the first ear.

16. The method of claim 15 wherein the light energy is applied in a continuous sweeping motion using a laser device that emits a line of laser energy.

17. The method of claim 15 wherein the light energy comprises light energy at 405 nm.

18. The method of claim 15 wherein the light energy consists of light at 405 nm±10 nm.

19. The method of claim 15 where the light energy is applied for 3-5 minutes.

20. The method of claim 15 further comprising applying visible light energy between 400-500 nm externally to himself from about one inch above his second ear down the side of his neck below the second ear.
